(19) United States
(12) Patent Application Publication
(10) Pub. No.: US 2009/0149831 A1
(43) Pub. Date: Jun. 11, 2009

(54) Polymeric Microspheres for Treatment of Obesity

(76) Inventors: Goldi Kaul, Nyack, NY (US); Katie L. Krueger, Merrimack, NH (US)

Correspondence Address: FAY KAPLUN & MARCIN, LLP, 150 BROADWAY, SUITE 702, NEW YORK, NY, 10038, US

(21) Appl. No.: 12/255,271

(22) Filed: Oct. 21, 2008

Related U.S. Application Data
(60) Provisional application No. 60/992,483, filed on Dec. 5, 2007.

(51) Int. Cl. A61K 9/50; A61M 5/31; A61K 47/02; A61B 1/018; A61P 3/04

(52) U.S. Cl. 604/511; 604/59; 424/489; 514/769; 600/104

(57) ABSTRACT

A system for treating obesity, comprises an injectable compound for intramuscular administration in a pyloric sphincter, the injectable compound including a plurality of microspheres and a fluid carrier in which the microspheres are suspended. The system further comprises an injection device having flexibility sufficient to pass through a working channel of an endoscope into the stomach to an injection location adjacent to the pyloric sphincter, the injection device including a tissue piercing tip and a lumen through which the injectable compound may be delivered.

### PRIORITY CLAIM

This application claims the priority to the U.S. Provisional Application Ser. No. 60/992,483, entitled “Polymeric Microspheres for Treatment of Obesity” filed Dec. 5, 2007. The specification of the above-identified application is incorporated herewith by reference.

### BACKGROUND

Surgical treatments for morbid obesity are often necessary when approaches such as lifestyle changes such as reduced calorie intake with or without appetite suppressing medication and/or increased exercise are unsuccessful. These procedures may include gastric bypass such as the Roux-En-Y procedure, gastric banding, implantation of stimulation devices, or placing mechanical restrictions in the stomach. Many of the approaches reduce the effective size of the stomach, fill part of the stomach with a restriction, or artificially cause the patient to feel full. Alternatively, the flow of food into the intestines is reduced, so that fewer calories are absorbed. Surgical methods generally reduce caloric intake by either impeding absorption of ingested calories or reducing the amount of food required to cause feelings of satiety.

Current surgical treatments for obesity often involve invasive, open surgery which is painful and which may entail serious side effects and significant recovery times. Even at expert centers dedicated to carrying out these procedures, the mortality rate may be approximately 0.5%. Additional drawbacks include staple and/or suture line leakage, ulcers forming at gastrojejunal anastomoses, long term nutritional deficiencies, port problems, band slipping, and band erosion.

### SUMMARY OF THE INVENTION

Described herein according to one exemplary embodiment is a system for treating obesity, comprising an injectable compound for intramuscular administration in a pylorus, microspheres of the injectable compound for altering the compliance and reducing a lumen area of the pyloric sphincter, a fluid carrier of the injectable compound for transporting the microspheres, and an injection device to administer the injectable compound.

Another exemplary embodiment is directed to an injectable compound for treating obesity, comprising a fluid carrier injectable intramuscularly into a pylorus, and microspheres transported in the fluid carrier to alter compliance and reduce a lumen area of the pyloric sphincter.

### BRIEF DESCRIPTION OF THE DRAWINGS

FIG. 1 is a diagram showing a side view of a stomach with the pyloric sphincter where the procedure according to the present invention is carried out; and

FIG. 2 is a cross sectional view on line II-II showing the pyloric sphincter of FIG. 1 and an injection device according to the present invention.

### DETAILED DESCRIPTION

The present invention may be further understood with reference to the following description and to the appended drawings, wherein like elements are referred to with the same reference numerals. The present invention relates to methods and devices' for treating morbid obesity. In particular, the present invention relates to changing the properties of the pyloric sphincter by introducing a therapeutic agent thereinto to delay gastric emptying, expedite satiety and decrease food consumption. Although the application describes the injection of microspheres, any of a variety of known bulking agents may be used to tighten the pyloric sphincter.

The embodiments of the present invention provide methods and systems to treat obesity by delaying the emptying of contents of the stomach into the intestines, thus leading to early and prolonged satiety and reduced food intake. The exemplary procedures are less invasive than prior obesity treatments as they may be limited to the injection of a therapeutic compound containing microspheres into the pyloric muscle. More specifically, in one exemplary embodiment of the present invention, the microspheres are administered intramuscularly into the pyloric muscle in the vicinity of the pyloric sphincter to affect the behavior of the sphincter.

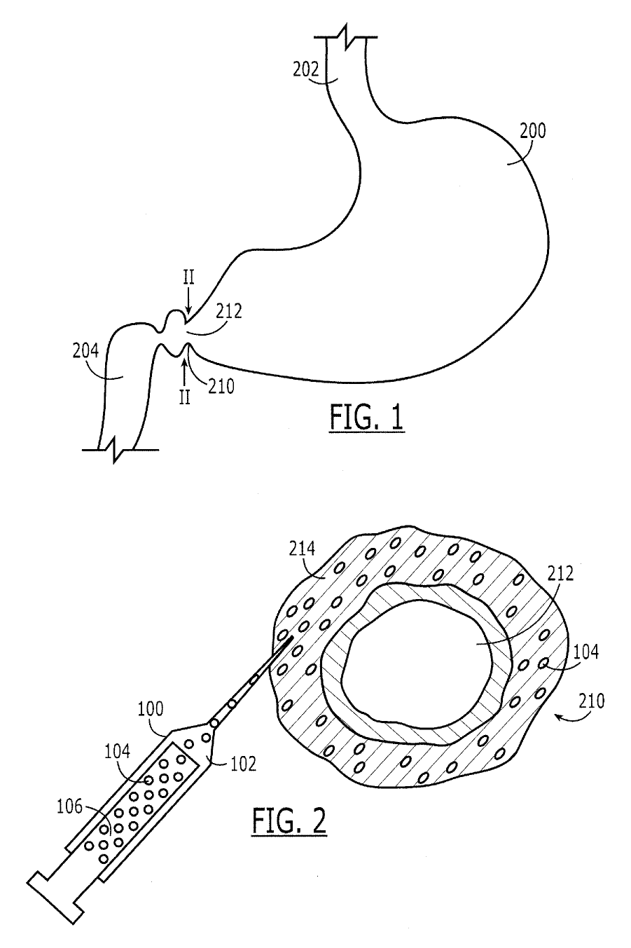

As shown in FIGS. 1 and 2, the distal end of the stomach 200 is connected to the duodenum 204 by the pylorus 214 which is separated from the stomach by the pyloric sphincter 210. Partially digested food, or chyme, passes through the pyloric sphincter 210 to continue digestion in the intestines. By controlling the operation of the pyloric sphincter 210 it is possible to control the rate at which food passes into the intestines. Retaining food in the stomach 200 longer reduces the rate at which space in the stomach 200 can be freed for more food and brings on satiety sooner.

In an exemplary embodiment, multiple polymeric microspheres 104 may be injected into the pyloric sphincter 210 to alter the compliance of the sphincter 210. The microspheres 104 may be combined with a carrying fluid (e.g., hyaluronic acid, saline solution, etc.) to form an injectable compound 106. The polymeric microspheres 104 are preferably selected to have dimensions and properties of compressibility and rigidity so that the injectable compound 106 generates desired changes on the tissue into which it is injected. For example, the microspheres 104 will preferably have a compressibility and a rigidity sufficient to withstand peristaltic movements of the GI tract and to reduce the compliance of the pyloric sphincter 210 to a desired degree. The total injection volume may vary between approximately 0.25 ml and 30 ml. The number of microspheres used for the procedure would depend on the condition and the nature of narrowing that the physician wants to achieve. The density of the microspheres would depend on the manner in which the microspheres are prepared. Porous microspheres may be prepared having lower densities than their non-porous counterparts. Densities of the microspheres would typically range from 0.2-1.5 g/cc. Skeletal density of the microspheres would also depend on the polymer type used for their synthesis. For this particular application, two more important properties are compressibility and rigidity of the microspheres. A preferred implementation incorporates a rigid microsphere that compresses sufficiently enough to be delivered through a needle but at the same time retains its inherent shape and size after delivery into the muscle.

Examples of suitable non-degradable polymers include polyhydroxyl methacrylates (polyHEMAs), carbohydrates, polyacrylic acids, polymethacrylic acids, polyvinyl sulfonates, carboxymethyl celluloses, hydroxyethyl celluloses, substituted celluloses, polyacrylamides, polyamides, polyureas, polyurethanes, polyesters, polyethers, polysaccharides, polylactic acids, polymethylmethacrylates, polycaprolactones, polyglycolic acids, polylactic-co-glycolic acids (e.g., polyd-lactic-co-glycolic acids) and copolymers or mixtures thereof. Examples of biodegradable polymers include PLAs, PGAs, polycaprolactones (e.g., poly-M-caprolactone), polyglycolic acids, polylactic-co-glycolic acids (e.g., polyd-lactic-co-glycolic acids, poly lactic acid (e.g., poly-L-lactic acid, poly-D,L-lactic acid), poly-p-dioxanones, polytri-methylene carbonates, polyanhydrides, polyortho esters, polyurethanes, polyamino acids, polyhydroxy alcanoates, polyphosphazenes, poly-b-malein acids, collagen (proteins), chitin, chitosan (polysaccharides), fibrin and albumin. Examples of techniques used to make suitable microspheres include methods shown in Tables I and II below.

TABLE I
Chemical Processes for Microsphere fabrication
Chemical processes

| S. No | Process Type (Polymer) | Suspending medium |
|---|---|---|
| 1. | Complex coacervation | Water |
| | (Water soluble | |
| | polyelectrolyte) | |
| 2. | Coacervation by polymer- | Organic solvent |
| | polymer incompatibility | |
| | (Hydrophilic or hydrophobic | |
| | polymers) | |
| 3. | Interfacial polymerization at | Aqueous/organic solvent |
| | liquid-liquid and solid- | |
| | liquid interfaces | |
| | (Water soluble and insoluble | |
| | monomers) | |
| 4. | In situ polymerization | Aqueous/organic solvent |
| | (Water soluble and insoluble | |
| | monomers) | |
| 5. | Solvent evaporation or in- | Aqueous/organic solvent |
| | liquid drying | |
| | (Hydrophilic or hydrophobic | |
| | polymers) | |
| 6. | Thermal or ionic gelation | Organic |
| | (Hydrophilic or hydrophobic | |
| | polymers) | |
| 7. | Desolvation in liquid media | Aqueous/organic solvent |
| | (Hydrophilic or hydrophobic | |
| | polymers) | |
| 8. | Super critical fluid | Aqueous/organic solvent |
| | technology | |

TABLE II
Mechanical Processes for Microsphere fabrication
Mechanical Processes

| S. No. | Process Type (Polymer) |
|---|---|
| 1. | Spray drying |
| | (Hydrophilic or hydrophobic polymers) |
| 2. | Spray chilling |
| | (Hydrophilic or hydrophobic polymers) |
| 3. | Fluidized bed drying |
| | (Hydrophilic or hydrophobic polymers) |
| 4. | Electrostatic deposition |
| 5. | Centrifugal extrusion |
| 6. | Interfacial polymerization at solid-gas or liquid-gas interfaces |
| 7. | Spinning disk |
| 8. | Extrusion or spraying into a desolvation bath |

As described above, by altering the compliance and size of opening of the pyloric sphincter 210, gastric emptying of the chyme into the duodenum 204 is restricted retaining increased volumes of food in the stomach 200 and expediting and prolonging satiety. That is, the injected polymeric microspheres 104 not only increase the resistance of the sphincter 210 to opening, they also bulk the tissue of the pyloric muscle 214 reducing an area of a lumen 212 through the sphincter 210.

The polymeric microspheres 104 may be administered to the patient through an intramuscular injection into the circular and/or longitudinal pyloric muscle 214, for example using an injection device 102 which may be inserted into the stomach 200 via the esophagus using an endoscope (not shown) as would be understood by those skilled in the art. The injection device 102 may, for example, be similar to a sclerotherapy needle which would be compatible with a conventional syringe. The distal tip of the device would incorporate a needle for penetrating the pyloric wall from the inner lumen of the GI tract.

Alternatively the microspheres may be injected laproscopically. An injection device could be delivered through the laparoscopic port and the penetrating needle of the device would enter the external surface of the pyloric wall to deliver the microspheres.

Those of skill in the art will understand that the spatial placement of the microspheres 104 into the pylorus 214 may be varied to suit different applications and to obtain desired therapeutic effects. For example, the microspheres 104 may be administered as multiple boluses injected at a plurality of locations spaced circumferentially around the pyloric muscle 214. Alternatively, the microspheres 104 may be injected as a single bolus in one location to localize the reduced compliance at a desired location radially around the sphincter 210 and/or to generate a portion of the sphincter 210 which projects into the lumen 212 at the radial location.

In another embodiment according to the invention, the microspheres 104 may provide additional functionalities. For example, radiopaque elements or a fluoroprobe may be included in the injectable compound 106 or as part of the microspheres 104 to facilitate visualization of the deployed microspheres 106 within the pylorus 214 using a fluoroscope, an endoscope and/or a CT scanner. Visual markers may also be included in all or a portion of the microspheres 104, to permit visual observation of the deployment pattern.

One or more therapeutic agents may be added to the microspheres 104 or to the injectable compound 106 for treatment of the tissue into which the microspheres 104 are injected. For example, the microspheres 104 may be coated with a therapeutic agent which facilitates the generation of satiety signals or which reduces the number of calories extracted from ingested food as would be understood by those skilled in the art. Alternatively or additionally, the therapeutic agent may be added to the carrying fluid which, along with the microspheres 104, forms the injectable compound 106. As would be understood by those skilled in the art, other therapeutic agents may be added to the injectable compound 106 as necessary to achieve therapeutic goals. Furthermore, the exemplary microspheres 104 may be formed to exhibit bio-adhesive properties to enhance their attachment to the tissue of the pyloric muscle 214 making migration of the microspheres 104 away from the injection location less likely. For example, a bio-adhesive coating may be provided including any of polymers such as poly acrylic acid, polyethylene glycol, polyN-vinyl, 2-pyrollidone, hyaluronic acid, hydroxyethyl cellulose, methylcellulose, pectin, carboxy methyl cellulose, alginates, chitosan, gelatin, dextrans etc. Alternatively, the microspheres may be coated with a material promoting scarring to tighten the sphincter to promote fibrin encapsulation or mucal deposits to further tighten the sphincter.

The system and method according to the present invention allows the microspheres 104 to be delivered directly into muscle (such as the pyloric muscle 214) to enhance packing while reducing slip planes which allows users greater control of the type and degree of the alteration in the properties of the pyloric sphincter 210. The substantially spherical shape of the microspheres 104 reduce muscle trauma achieving a corresponding reduction in discomfort and side effects as would be understood by those skilled in the art. In addition, as the sphincter 210 is accessed via an endoscope inserted via a naturally occurring body orifice (i.e., the mouth) the only penetration of tissue required is the piercing of the sphincter 210 by the injection device 102. Thus, the discomfort, complications and extended recovery times associated with open surgery are avoided.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A system for treating obesity, comprising:
an injectable compound for intramuscular administration in a pyloric sphincter, the injectable compound including:
a plurality of microspheres; and
a fluid carrier in which the microspheres are suspended; and
an injection device including a distal portion adapted for insertion to a target injection site one of adjacent to and in the pyloric sphincter.

2. The system according to claim 1, wherein the injection device has a flexibility sufficient to pass through a natural body lumen into the stomach to the target injection site.

3. The system according to claim l,wherein the microspheres are polymeric microspheres.

4. The system according to claim 1, wherein the injection device is sized to be slidably received in a working channel of one of an endoscope and a laparascope.

5. The system according to claim 1, wherein the injection device includes a tissue piercing tip and a lumen through which the injectable compound may be delivered.

6. The system according to claim 1, wherein the microspheres have compressibility and rigidity sufficient to withstand peristaltic movement of the GI tract.

7. The system according to claim 1, wherein the microspheres have diameters between about 100 microns and 5,000 microns.

8. The system according to claim 1, wherein the fluid carrier comprises one of saline and hyaluronic acid.

9. The system according to claim 1, wherein the injection device comprises a syringe.

10. The system according to claim 1, wherein the injectable compound is administered as a single bolus.

11. The system according to claim 1, wherein the injectable compound is administered as multiple boluses.

12. The system according to claim 1, further comprising at least one of a radiopaque element, a fluoroprobe and a visual marker of the injectable compound.

13. The system according to claim 1, wherein outer surfaces of the microspheres comprise a bio-adhesive.

14. The system according to claim 1, wherein the injectable compound comprises an agent for treatment of obesity.

15. An injectable compound for treating obesity, comprising:
a fluid carrier injectable intramuscularly into one of a pyloric sphincter and tissue adjacent to a pyloric sphincter; and
microspheres suspended in the fluid carrier to alter one of a compliance of the pyloric sphincter and a lumen area of the pyloric sphincter.

16. The injectable compound according to claim 15, wherein the microspheres include at least one of a radiopaque element, a fluoroprobe, and a visual marker.

17. The injectable compound according to claim 15, wherein the microspheres include an agent for treatment of obesity.

18. The injectable compound according to claim 15, wherein the microspheres comprise a bio adhesive.

19. The injectable compound according to claim 15, wherein the microspheres are polymeric microspheres.

20. The injectable compound according to claim 15, wherein the fluid carrier comprises one of saline and hyaluronic acid.

21. The injectable compound according to claim 15, wherein the microspheres have a density of about 0.2 g/cc to 1.5 g/cc.

22. The injectable compound according to claim 15, wherein the microspheres have diameters between about 100 microns and 5,000 microns.

23. A method for treating obesity, comprising:
inserting an injection apparatus to an injection location one of adjacent to and in a pylorus of a living body; and
injecting into tissue of the pylorus a bulking agent including a plurality of microspheres to alter one of a compliance and a lumen size of a pyloric sphincter of the pylorus.

24. The method according to claim 23, wherein the injection apparatus is inserted to the injection location through a body lumen accessed via a naturally occurring body orifice

25. The method according to claim 23, wherein the bulking agent includes a fluid carrier.

26. The method according to claim 24, wherein the injection apparatus is inserted to the injection location through a working channel of an endoscope.

27. The method according to claim 23, wherein the step of injecting is performed using a syringe.
